(19) United States
(12) Patent Application Publication
(10) Pub. No.: US 2005/0240202 A1
(43) Pub. Date: Oct. 27, 2005

(54) Devices and methods of repairing cardiac valves

(76) Inventors: Hani Shennib, Montreal (CA); Doug Rowe, San Jose, CA (US); Joshua Stafford, Menlo Park, CA (US); John Barrett, San Francisco, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/830,489

(22) Filed: Apr. 21, 2004

(51) Int. Cl. A61B 17/10

(52) U.S. Cl. 606/142

(57) ABSTRACT

Devices and methods are provided for temporarily and permanently apposing together leaflets of a cardiac valve.

### FIELD OF THE INVENTION

The invention relates to devices and methods for the less invasive repair of cardiac valves, and particularly to less invasive repair of mitral and tricuspid valves.

### BACKGROUND OF THE INVENTION

The human heart has four valves; the aortic valve, the mitral valve, the pulmonary valve and the tricuspid valve. Various diseases and certain genetic defects of the heart valves can impair the proper functioning of the valves. Improper functioning of a valve can be severely debilitating and even fatal if left untreated, particularly if the diseased valve is the aortic valve (between the left ventricle and the aorta) or the mitral valve (between the left atrium and left ventricle). The common defects and diseases affecting each of these valves, and the treatments thereof, are typically different.

The aortic valve and, infrequently, the pulmonary valve, are prone to stenosis. Stenosis typically involves the buildup of calcified material on the valve leaflets, causing them to thicken and impairing their ability to fully open to permit adequate forward blood flow. Because stenotic damaged sustained by leaflets is irreversible, the most conventional treatment for stenotic aortic and pulmonic valves is the removal and replacement of the diseased valve.

On the other hand, the mitral valve and, less frequently, the tricuspid valve, are more prone to deformation, such as dilation of the valve annulus, tearing of the chordae tendinae and leaflet prolapse, which results in valvular insufficiency wherein the valve does not close properly and allows for regurgitation or back flow from the left ventricle into the left atrium. Deformations in the structure or shape of the mitral or tricuspid valve are repairable. Thus, because prosthetic valves have certain disadvantages that can have serious effects (e.g., mechanical valves carry the risk of thromboembolism and require anticoagulation treatment, and biological valves have limited durability), an improper functioning mitral or tricuspid valve is ideally repaired rather than replaced.

The mitral valve includes two leaflets or cusps, called the anterior and posterior leaflets, which are encircled by a dense fibrous ring of tissue known as the annulus. The leaflets are of unequal size with the posterior leaflet having a wider attachment area to the annulus. The end of the lines at which the leaflets come together are called the commissures. The leaflets are held in place by the chordae or threads connected at the base by two papillary muscles which extend from the underside of the leaflets to the papillary muscles within the wall of the left ventricle. The annulus of a normal mitral valve is somewhat “D” shaped.

The tricuspid valve, also an atrioventricular valve, functions similarly to the mitral valve but has three leaflets rather than two. The three leaflets, referred to as the anterior, posterior, and septal leaflets, and are roughly triangular in shape. Like the mitral valve leaflets, the tricuspid valve leaflets are encircled by a fibrous annulus and are held in place by chordae connected to associated papillary muscles. The annulus of the tricuspid valve is more nearly circular than is the mitral valve. While the two valves function very similarly, the mitral valve is subject to significantly higher back pressure than is the tricuspid valve and, as such, the. mitral valve is more susceptible to degradation and deformation.

During systolic contraction of the heart, the free margins of the mitral leaflets and tricuspid leaflets, respectively, come in apposition to each other and close the respective atrial-ventricular passage. The chordae and papillary muscles hold the leaflets in this position throughout the systole cycle to prevent the leaflets from bulging into and opening within the associated atrium. However, when the valve or its leaflets are misshapen or enlarged, for example, when the annulus is dilated, the edges of the leaflets fail to meet each other, leaving an opening there between. This opening may involve lateral separation of the valve leaflets and/or elevation of one valve leaflet with respect to the other. In either case, the ineffective closure of the valve during ventricular contraction results in regurgitation or leakage of blood back into the atrium during ventricular contraction, and ultimately in reduced pumping efficiency. To compensate for such inefficiency in the mitral valve, for example, the left ventricle must work harder to maintain the requisite cardiac output. Overtime, this compensatory mechanism typically results in hypertrophy of the heart followed by dilation, i.e., an enlarged heart, which can lead to congestive heart failure.

Any one or combination of the annulus, the leaflets, the chordae and the papillary muscles may be the cause of the mitral and/or tricuspid insufficiency and/or regurgitation. Common conditions or diseases to the mitral and tricuspid valves which may result in mitral regurgitation include dilation of the annulus, ischemic regurgitation and myxomatous degeneration of the valve leaflet. Annular dilation typically involves the elongation or dilation of the posterior two-thirds of the mitral valve annulus, the section corresponding to the posterior leaflet. Ischemic regurgitation involves a lack of blood supply to the valve tissue, particularly the papillary muscles, due to coronary artery disease. Myxomatous degeneration involves weakness in the leaflet structure, leading to thinning of the tissue and loss of copation.

Various surgical techniques may be used to repair diseased or damaged mitral and tricuspid valves. These include but are not limited to annuloplasty (i.e., contracting the valve annulus to restore the proper size and shape of the valve), quadrangular resection of the leaflets (i.e., removing tissue from enlarged or misshapen leaflets), commissurotomy (i.e., cutting the valve commissures to separate the valve leaflets), shortening and transposition of the chordae tendonae, reattachment of severed chordae tendonae or papillary muscle tissue, and decalcification of valve and annulus tissue.

Another repair technique, commonly referred to as “bow-tie” repair, involves the edge-to-edge suturing together of the anterior and posterior leaflets. Typically, at least one suture is placed centrally with respect to the commissure line, creating a double orifice valve, thereby preventing prolapse at the central portions of the leaflets and reducing or eliminating regurgitation. The sutures may alternatively or additionally be placed closer to the commissures. These steps are typically performed using arrested, open heart techniques. Following the valve repair procedure, ultrasound is typically used to verify the repair.

Because they are performed on stopped hearts through an open chest approach, conventional valve repair techniques may require minimal instrumentation and time. However, because the success of the repair can only be tested on a beating heart, the heart must be closed up and the patient taken off the heart lung machine before testing can be done. If the repair is determined to be inadequate, the patient must be put back on cardiopulmonary bypass and the heart must be reopened.

Moreover, the risks and complications associated with open-heart surgery, which involves the use of cardiopulmonary bypass, aortic cross-clamping and cardioplegia arrest, are well known. The most serious risks of cardiopulmonary bypass and aortic cross-clamping are the increase in the likelihood of bleeding and stroke. Also, patients who undergo surgeries using cardiopulmonary bypass often require extended hospital stays and experience lengthy recoveries. Thus, while conventional heart valve surgery produces beneficial results for many patients, numerous others who might benefit from such surgery are unable or unwilling to undergo the trauma and risks of conventional procedures.

Within recent years, minimally invasive types of procedures for coronary artery bypass surgery have been developed which do not require stopping the patient's heart and the use of cardiopulmonary bypass; however, no such minimally invasive surgical procedure has been developed for the repair of cardiac valves.

Thus, it is desirable to provide a device which, when operatively used, involves a simplified procedure by which to repair a cardiac valve, in particular, mitral and tricuspid valves. For example, it would be beneficial to provide a device which, when properly implanted, corrects a defective valve in addition to other co-morbidities affecting proper function of the valve, obviating the need to perform ancillary procedures to correct leaflet size and shape, to adequately coapt the leaflets, to reattach or shorten chordae, etc. In addition, it is desirable to provide a valve repair procedure which requires minimal instrumentation and steps, is easier to perform than conventional valve repair procedures and reduces the time and cost of the procedure. Moreover, it is desirable to provide a valve repair procedure that obviates the need for cardiopulmonary bypass, can be performed on a beating heart, involves endovascular or less invasive techniques, can be performed on a patient while awake and/or in an ambulatory setting by surgeons, cardiologists or interventionalists.

### SUMMARY OF THE INVENTION

The present invention includes devices, methods and kits for repairing cardiac valves, particularly mitral and tricuspid valves experiencing regurgitation. The devices of the present invention provide various functions including one or more of temporarily capturing, grasping or snaring one or more of the chordae tendinae and/or leaflets, using the captured chordae as a guide to reaching the leaflets, appositioning the leaflet edges, stabilizing or immobilizing the natural motion of the valve leaflets, evaluating or verifying the effectiveness of one or more selected points of apposition between the leaflets, measuring the flow and/or pressure gradient characteristics of a valve, and/or permanently clipping or affixing the captured chordae wherein the valve leaflets are permanently apposed at one or more points along the leaflet edge or commissure line. Each of these functions may be provided in a stand alone device or two or more functions maybe combined as an integral part of the same device.

The devices of the present invention may be configured to utilize a natural tissue structure, such as the chordae tendinae, or another structure, such as a guide wire, to position their distal working ends at a point along one or more leaflet edges. As such, the devices are designed to track along one or more chordae or a guide to a site of intended stabilization, immobilization and/or apposition of a valve leaflet.

In certain variations of the invention, a device provides a chordae capturing mechanism for temporarily capturing or snaring one or more chordae, more typically two or more chordae, and collecting or synching them together so as to provide a means for guiding or tracking the distal end of the device there over or there along to reach the valve's leaflets. Preferably, at least two chordae, i.e., at least one chordae of each of two opposing valve leaflets, are synched together at a location along their length proximate to the underside of the leaflet edges wherein such proximity defines a point of apposition between the leaflet edges and/or stabilizes a motion of a portion of the leaflets, particularly at that point of apposition. The same device or another device may provide a means for delivering and applying or deploying a mechanism or a means for clipping, fastening or otherwise permanently affixing the collected chordae together at the selected point of apposition so as to permanently maintain apposition of the leaflets at the selected point of apposition.

The subject devices may also be configured for temporarily capturing the valve leaflets. The valve leaflets may be captured in such a way so as to apposition adjacent valve leaflets along their edges, i.e., along the commissure line, and or to stabilize or immobilize at least a portion of one or more leaflets. A dimension of the leaflet capturing device may be selected based on the size of the intended area of the leaflet(s) to be stabilized. The same device or another device may provide a means for delivering a leaflet fastening or clipping mechanism for permanently fastening or attaching together two or more leaflets. The fastening mechanism may be employed while the leaflet edges are appositioned and/or stabilized by the leaflet capturing mechanism.

Any one of the above mentioned devices or another device may provide a means for evaluating or verifying the effectiveness of apposing the leaflets at any one selected point of apposition between the leaflets or between chordae of opposing leaflets. In a particular embodiment, a device assesses the effect of leaflet apposition on the pressure gradient between the atrium and ventricle. As mentioned above, the subject assembly may further include one or more means for evaluating or verifying the effectiveness of the one or more selected points of apposition prior to permanent placement of the fastener. Such evaluating or verification means may include pressure monitoring probes or components for measuring the pressures just above and just below the valve leaflets, i.e., in the atrium and the ventricle, respectively, and for determining the pressure gradient or differential there between. Additionally or alternatively, one or more flow monitoring probes may be included for measuring the normal flow and back flow of blood through the valve.

The devices may have features and mechanisms which assist in their own delivery to a target location at a valve leaflet as well as assist in the delivery of other devices to the chordae and/or valve leaflet(s). In certain embodiments, the devices are configured to be delivered from within the heart chamber to the valve leaflet, for example, from within the ventricle to the valve leaflet(s).

The present invention further provides fastening mechanisms to be attached to a valve or subvalvular component, such as a valve leaflet or chordae tendinae, and in particular to fix together adjacent chordae or valve leaflets at one or more apposition points. Any suitable fastener may be used with the present invention including, but not limited to clips, staples, coils, buttons, sutures and the like. The fasteners may be made of biodegradable or non-biodegradable materials as well as those materials which are inert and non-thrombogenic. The subject devices may further include a means for anchoring the fastener or clip to an appropriate location on the cardiac anatomy to prevent embolization of the fastener in case it becomes unattached from the chordae and/or valve leaflets.

One or more of the above devices may be provided as part of an assembly for delivering, positioning and fastening or implanting the fastener or clip. The subject devices may be configured for less invasive surgical and endovascular approaches, wherein the implantable fastener and associated chordae and/or leaflet capturing, stabilizing, appositioning, fastening and flow/pressure evaluation components are provided as part of a cannula or catheter assembly, respectively. As such, the implantable devices, flow probes and/or pressure monitors are configured for delivery through a cannula or catheter, or are themselves part of a cannula or catheter assembly.

The subject methods generally include one or more of delivering an implantable fastener or clip to a valve to be repaired; monitoring the blood flow characteristics and/or pressure gradient at the valve; capturing the chordae tendinae; using the chordae tendinae to locate a position at or under a valve leaflet; capturing at least a portion of one or more valve leaflets; appositioning the valve leaflets at one or more points along the leaflets, such as by grasping together the valve leaflets at a selected point along the commissure line; stabilizing or immobilizing at least a portion of one or more valve leaflets; determining, from monitoring the flow and/or pressure gradient characteristics or by visually evaluating with transesophageal echo (TEE), whether appositioning or grasping at such selected point improves or optimizes the flow characteristics and/or pressure gradient, i.e., reduces regurgitation through the valve; and fastening the captured chordae tendinae and/or valve leaflets at one or more selected points wherein the flow/pressure characteristics of the subject valve are improved or optimized. The subject methods may further include anchoring the fastener to an appropriate location of the cardiac anatomy in order to prevent embolization of the fastener in case it becomes unattached from the valve leaflets.

Such methods may further include repeating the steps of grasping or capturing the chordae tendinae and/or the leaflets, monitoring the blood flow characteristics and/or pressure gradient and determining whether the flow/pressure characteristics for each grasping step results in improvement or optimization in such flow/ pressure characteristics. The above described steps of grasping, capturing, and assessing flow and/or pressure may be repeated until one or more suitable apposition points are found, at which point(s) a fastener is locked into place onto the chordae and/or valve leaflets. As such, such methods further include the step of releasing the chordae and/or valve leaflets after the step of grasping the chordae and/or valve leaflets, upon a determination that there is insufficient or no improvement. Alternatively, the chordae and/or leaflets may be successively captured or grasped (with or without subsequent release) and fastened together at more than one selected location, i.e., two or more of the subject fasteners are permanently attached to the chordae tendinae and/or valve leaflets, until sufficient improvement in flow and or pressure characteristics are achieved.

These and other features and advantages of the invention will become apparent to those persons skilled in the art upon reading the details of the subject devices and methods as more fully described below.

### BRIEF DESCRIPTION OF THE DRAWINGS

The following drawings are provided and referred to throughout the following description, wherein like reference numbers refer to like components throughout the drawings:

FIG. 1A is a top view of an insufficient or defective mitral valve having leaflets which do not coapt with each other, resulting in regurgitation of blood from the ventricle into the atrium.

FIG. 1B is a cross-sectional view of the left side of the human heart, including the defective or insufficient mitral valve illustrated in FIG. 1A.

FIGS. 2A, 2B and 2C illustrate one variation of a device of the present invention for repairing regurgitant cardiac valves.

FIGS. 3A-3E illustrate another variation of a device of the present invention.

FIGS. 4A-4D illustrate another embodiment of a device of the present invention.

FIGS. 5A and 5B illustrate a delivery sheath for use with the present invention.

### DETAILED DESCRIPTION OF THE INVENTION

As mentioned above, the present invention includes devices, methods and kits for repairing cardiac valves, particularly mitral and tricuspid valves experiencing regurgitation.

Before the present invention is described in detail, it is to be understood that this invention is not limited to particular embodiments and applications described, as such may, of course, vary. For example, the following description of the invention is primarily described in the context of mitral valve repair; however, such description, with certain obvious modifications to the invention, is also intended to apply to the repair of tricuspid valves as well as other tissue structures similar to that of cardiac valves. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

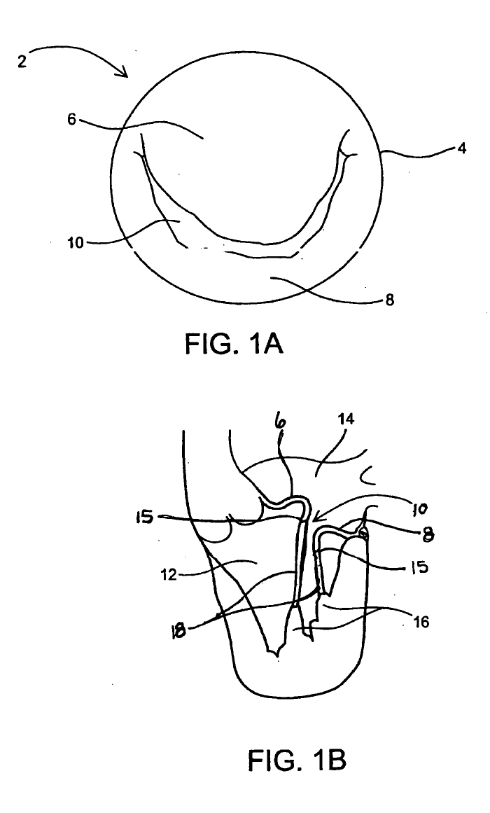

To better understand the present invention, FIGS. 1A and 1B are provided to illustrate a defective mitral valve 2 and the resulting effect on the functioning of the valve 2. Specifically, FIG. 1A illustrates a defective mitral valve 2 having an annulus 4, an anterior leaflet or cusp 6 and a posterior leaflet or cusp 8. Mitral valve 2 suffers from valvular insufficiency as evidenced by the gap 10 between the two leaflet edges during systole. FIG. 1B is a cross-sectional view of the left side of a heart having a left ventricle 12, a left atrium 14 and defective mitral valve 2 situated at the atrioventricular passageway there between. Mitral valve 2 is tethered to papillary muscles 16 by bundles of chordae tendinae 18. The cordae tendinae 18 extend from the papillary muscles 16 to attachment points 15 along the leaflet edges or commissure line.

FIG. 1B further illustrates the effect that the dilation of mitral valve 2 has on its ability to properly function as evidenced by gap 10 between the leaflet edges. Gap 10 may involve lateral separation of the valve leaflets and/or elevation of one valve leaflet with respect to the other. In all cases, the ineffective closure of the valve during ventricular contraction results in regurgitation or leakage of blood back into the atrium, thereby reducing the pumping efficiency of the heart during systole, i.e., reducing the amount of available oxygenated blood that is pumped by the left ventricle through the aortic valve to the body and brain.

The various embodiments of the devices of the present invention, which will now be described in detail, function to correct or improve the function of such a defective mitral valve 2. In further describing the present invention, devices of the present invention will be described first, followed by a description of the methods of using the subject device to temporarily or permanently fasten or clip leaflets of a valve together. Kits which include the subject devices will then be described.

The subject devices, methods and kits can be used to repair a variety of cardiac valves, wherein mitral valve repair applications will be used herein for exemplary purposes only, and is no way intended to limit the scope of the invention.

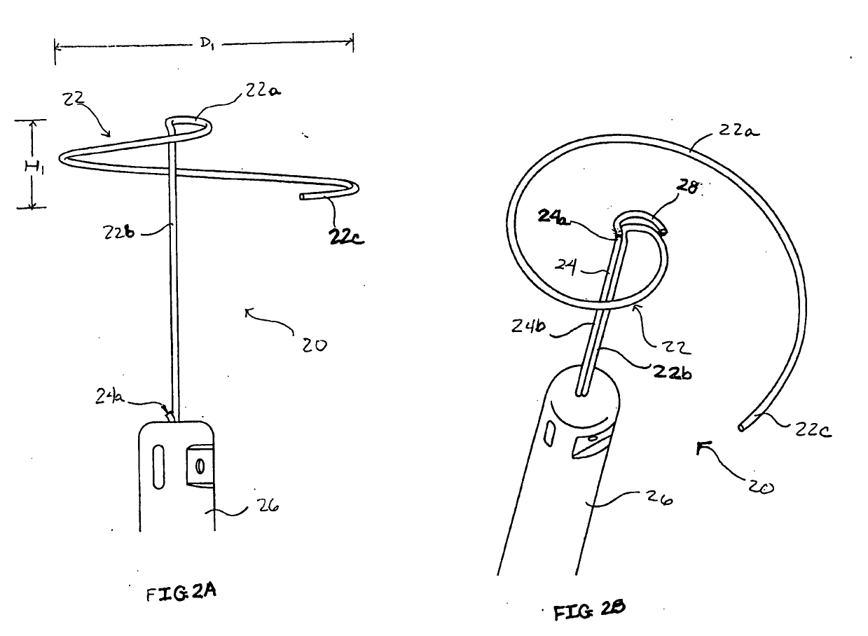

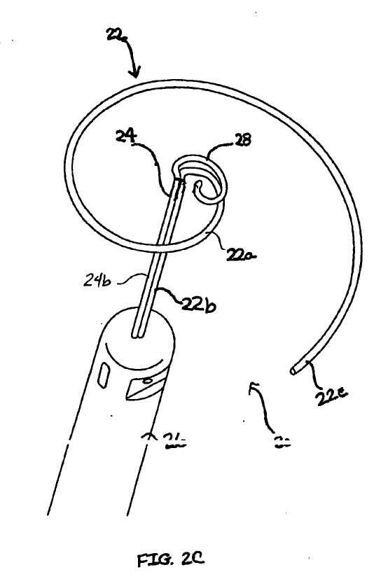

Referring now to FIGS. 2A, 2B and 2C, there is illustrated an exemplary embodiment of a device 20 of the present invention for repairing cardiac valves. Device 20 includes a tissue capturing, grasping or snaring member 22, which may also act as a guide member, and a fastening means application member 24, both of which distally extend from a main body 26, which may be a catheter, a cannula or the like. The two members are operated and employed cooperatively to accurately and safely deliver and deploy a fastening mechanism at a target valve site.

In the illustrated embodiment, capturing member 22 and application member 24 each have a thin wire or ribbon form, but may be differently configured. Capturing member 22 is configured at a distal working end 22a for capturing or grasping or snaring a natural tissue structure, e.g., chordae tendinae, leaflet edge, etc., or a prosthetic structure, e.g., a guide wire, selectively placed, and for guiding device 20 to a target valve to be repaired. Application or deployment member 24 is configured at a distal working end 24a to hold a fastening mechanism and deploy it at a target site on or near the target valve leaflets. Each of the members has an extension portion 22b, 24b which extends proximally into main body 26 and is controllable at a proximal end by the user. Extension portions 22b, 24b each have a substantially straight, axial configuration, and as such, may be positioned substantially parallel and in close proximity to each other to minimize the overall profile of device 20.

Working end 22a of capturing member 22 has at least one bend or turn to provide an open structure by which to capture or hold a tissue structure, such as chordae tendinae, or a prosthetic structure, such as a guide wire. In certain variations, working end 22a substantially defines a plane which is substantially perpendicular to the axis of extension portions 22b, but may be adjustable or canted to better accommodate the application at hand or the anatomy being treated. Working end 22a typically has an arcuate or annular configuration including but not limited to a hook, loop, spiral or coil configuration, but may alternatively have an angular configuration (e.g., triangular, square, etc.). In any case, the open configuration allows working end 22a to loop around a tissue structure or band, such as the chordae tendinae, or a guide wire or the like, and then track, translate or slide along the captured structure to a target tissue site.

In the illustrated embodiment, working end 22a is in the form of a spiral or coil terminating in a distal tip 22c and having a varying diameter D1. The spiral or coil may be slightly helical in nature to provide a height dimension H1. The height and diameter dimensions of working end 22a depend on the size (i.e., height and/or diameter) of the structure being captured or grasped. For mitral valve repair applications wherein capturing member 22 is used to capture or grasp the chordae tendinae, the diameter D1 of working end 22a ranges from about 10 mm to about 20 mm, where one embodiment might have a diameter ranging from about 12 mm to about 16 mm. The height H1 of working end 22a will generally range from about 0 to about 10 mm, where one embodiment might have a height in the range from about 4 mm to about 6 mm.

As mentioned above, working end 24a of clip deployment member 14 is configured to hold or grasp a fastening mechanism 28 during delivery of device 20 to the target valve and then deploy the fastening mechanism at a target site on the valve, such as on a leaflet(s) or on or about a subvalvular structure, e.g., chordae tendinae. More specifically, fastening mechanism 28 may be used to attach the edges of two opposing valve leaflets or to synch together chordae from each of both leaflets in order to provide a fixed or permanent apposition point between the two leaflets, and thereby correct for regurgitation caused by leaflet prolapse or the like.

As is described in greater detail below, any suitable fastening means may be employed, such as those used for surgical closure, valve surgery and anastomosis applications. One exemplary fastening means suitable for use with the present invention, and particularly with the embodiment of FIGS. 2A-2C, is Coalescent Surgical's U-CLIP™ Device as disclosed in U.S. Pat. No. 6,641,593. This device is a self-closing surgical clip made of Nitinol, which is movable between an open configuration and a closed configuration upon release from a mechanical restraining device, such as a coil or tubular wire, which holds the clip in its open configuration. Extension portion 24b may itself act as the restraining mechanism by having a lumen in which the clip is restrained until deployment, for example, by means of a pusher rod axially held within the lumen of extension portion 24b. Such a configuration allows for the successive delivery and deployment of clips without the need to remove device 20 or clip deployment member 24 from within the vasculature. A plurality of clips may be preloaded within the lumen of deployment member 24 or otherwise provided in a cartridge within main body 26. For applications in which the leaflet is to be captured, needles or other penetrating devices may be used with and provided integrally with device 20 to further facilitate application of the fastening means to the leaflets.

Either one or both of members 22 and 24 may be rotatable about the axis of their respective extension portions 22b, 24b. This ability provides for added flexibility and dexterity in reaching, grasping and fastening the chordae (or leaflets in certain cases), and obviates the need to rotate device 20 when positioned within the vasculature, thereby avoiding damage to the same. Additionally, each of members 22 and 24 may be axially moveable relative to the other and relative to main body 26 from a retracted or an unextended position to an extended position. FIGS. 2A, 2B and 2C each show guide member 22 in an extended position while application member 24 is shown in an unextended position in FIG. 2A, a partially extended position in FIG. 2B and a fully extended position in FIG. 2C. Device 20 may be configured such that the entirety of either or both the members is retractable within main body 26, which configuration provides a low profile to facilitate endovascular delivery of device 20. With such an embodiment, the fully-retractable member or working end thereof may be made of a malleable material or a shape memory material, such as Nitinol, wherein the working ends 22a, 24a have a straight, axial shape or unexpanded or low-profile configuration when in a fully retracted position within main body 26 and take on a preformed, expanded or working configuration when extended from the distal end of main body 26. Alternatively, one or both of members 22, 24 may be only partially retractable within main body 26. For example, where working end 22a of guide member 22 has a fixed configuration, only extension portion 22b is retractable within main body 26. It may be preferable, however, for at least one member to be at least partially retractable and extendable with respect to main body 26, and thus, at least partially moveable relative to the other member. For example, in the illustrated embodiment, at least application member 24 is retractable/extendable within main body 26 and movable relative to capturing member 22. As such, a fastening means may be held within main body 26 during delivery of the device to the target site so as to minimize the risk of dislodgment as well as to protect the anatomy through which device 20 is delivered. Capturing member 22 may also be retractable and made of a material which allows it to be in at least a partial axial configuration when retracted within main body 26 and then to morph into or return to its expanded, non-axial configuration when extended from main body 26.

In use, device 20 is delivered to the target valve and the distal end of main body 26 is positioned proximate the valve leaflets, most typically in a subvalvular position. Particularly in endovascular applications, device 20 is delivered with capturing member 22 and clip application member 24 retracted within main body 26. Capturing member 22 is then extended into an expanded condition and tip 22c is manipulated to select and/or separate one or more chordae from the others. Capturing member 22 is further manipulated, e.g., rotated, to gather the captured chordae towards the center of working end 22a. If necessary, capturing member 22 is translated, either by further extension of capturing member 22 or by advancement of main body 16, and tracked distally over the collected chordae to a location closer to the undersurface of the valve leaflets, thereby gathering opposing chordae together, and thus opposing leaflet edges attached to the captured chordae. The suitability of the resulting apposition is assessed, as described in greater detail below. If considered suitable, the working end 24a of fastener application member 24 is advanced toward working end 22a of capturing member 22 and a fastener is placed about the collected chordae. The procedure may be repeated as necessary to apply additional fasteners to effect additional apposition points between the leaflet edges.

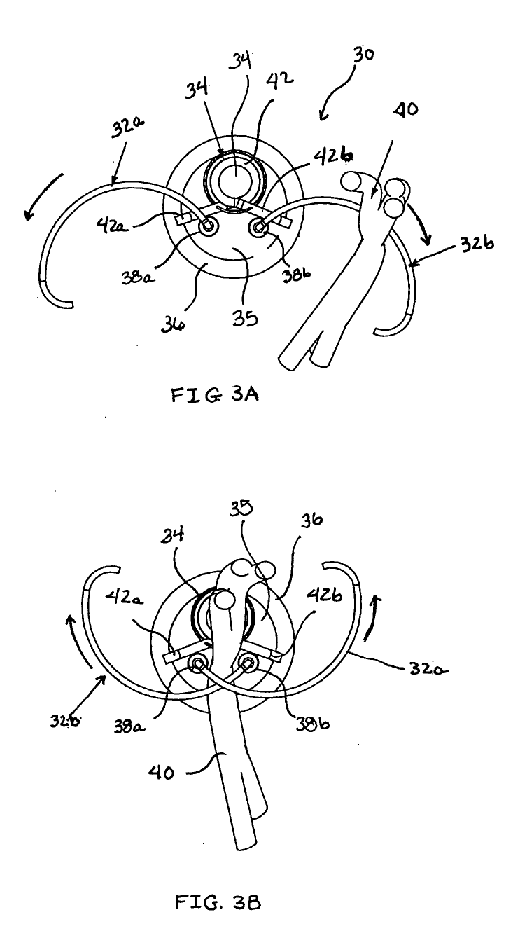

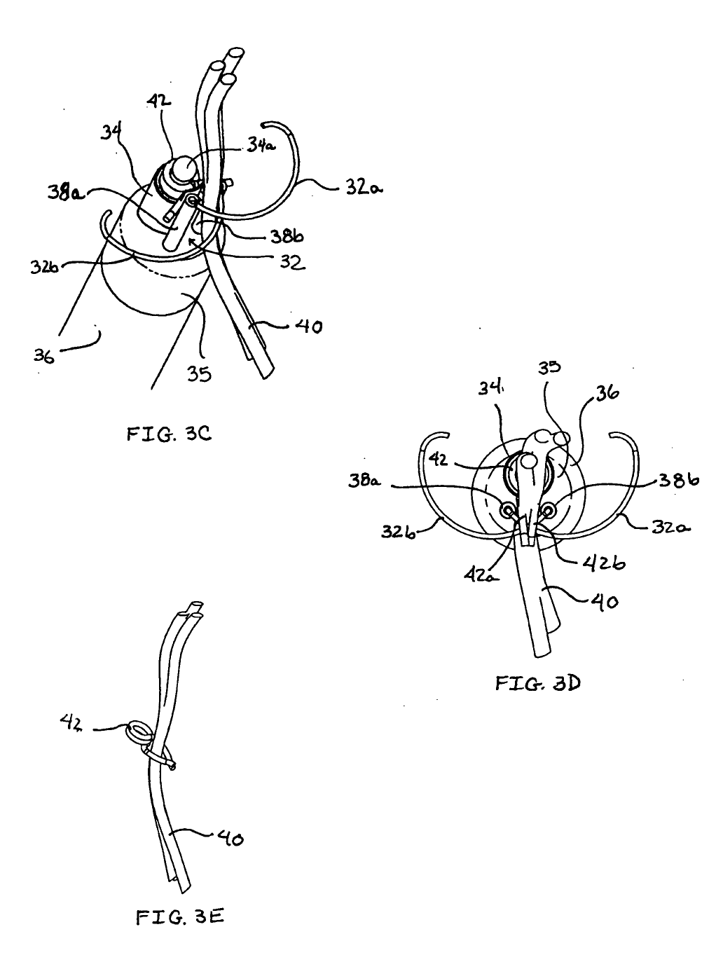

FIGS. 3A-3E illustrate another variation of a device of the present invention for repairing cardiac valves. A device 30 includes an inner or core member 35 axially extendable from and retractable within a main body or outer sheath or member 36, which may be a catheter, a cannula or the like. Extending from the distal end of core member 35 is capturing or guide member 32. Capturing member 32 includes rotatable arms 32a and 32b, each having an arcuate configuration, such as a semi-circular configuration, and positioned symmetrically with respect to each other. Each arm has an axial extension portion (not visible from the illustrated views) extending through an extension sleeve 38a, 38b which extends from the distal end of main body 36. Extension sleeves 38a and 38b provide rigidity and stability to arms 32a and 32b, respectively, when in use. In alternate embodiments arms 32a, 32b extend together through a single extension sleeve.

Arms 32a and 32b are controllable by the user at a proximal end of device 30 with each arm being rotatable about the axis defined by their respective post 38a, 38b. They may be independently operable from each other or be operated in tandem whereby, upon a single action, arms 32a and 32b rotate simultaneously but in opposite directions, i.e., arm 32a rotates in a counter-clockwise direction and arm 32b operated in a clockwise direction (from the viewpoint of FIGS. 3A and 3B) and visa-versa, so as to be selectively opened (see FIG. 3A) and closed (see FIGS. 3B-3D). In a closed position, arms 32a and 32b will “hug” a tissue structure 40, e.g., chordae tendinae, captured there between. The rotation is reversible so as to release the captured structure 40.

Arms 32a and 32b may also be retractable and extendable along their lengths, and made of a malleable or shape memory material to facilitate such, as described with respect to device 20 of FIGS. 2A-2C, wherein the arms have a constricted, unexpanded or low-profile configuration when retracted and have an expanded or working configuration when extended. For example, both arms may be fully retracted during delivery and removal of device 30 from the vasculature or anatomy to provide the advantages mentioned above. If not otherwise retracted, arms 32a and 32b are preferably in a closed positioned during delivery to a target site in order to reduce the profile of the device (see, e.g., FIG. 3B).

Device 30 also provides a clip application mechanism which functions to hold a clip prior to deployment and to apply or deploy the clip upon capturing the target tissue structure(s) 40. The particular configuration of this mechanism will vary according to the type of clip employed. For example, the illustrated embodiment is configured for holding and deploying a spring clip 42 configured to be biased closed wherein the legs 42a and 42b of clip 42. The clip application mechanism includes a retractable and extendable clip mount 34 at the distal end of device body 36 and a post or knob 34a extending distally from the clip mount. Alternatively, mount 34 may be fixed and knob 34a may be retractable and extendable. In use, clip 42 is loaded on clip mount 34 by placing the clip's loop in an open position over knob 34a. Extension of clip mount 34 or knob 34a beyond the distal end of arm posts 38a, 38b causes spring clip 42 to be released to its biased or closed state (as shown in FIG. 3E). In other embodiments extension posts 38a, 38b may be retractable and extendable simultaneously with arms 32a, 32b, thereby releasing the restraint on clip legs 42a, 42b allowing them to impinge on captured tissue structure 40, as shown in FIG. 3D. Alternatively, the extension portions of arms 32a, 32b, without posts 38a, 38b, may themselves serve to restrain and release the clip legs, however, the former design allows the arms to be formed from a smaller gauge wire, thereby maximizing their malleability. With any of these variations, clip mount 34 or knob 34a may then be retracted to fully disengage clip 42 from device 30.

In use, device 30 is delivered to the target valve preferably with inner member 35 retracted within outer sheath 36. Inner member 35 is extended beyond the distal end of main body 36 and positioned proximate the valve leaflets, most typically in a subvalvular position. Again, particularly in endovascular applications, it may be beneficial to have inner member 35 and grasping arms 32a and 32b in a fully retracted position upon delivery. Upon delivery, arms 32a and 32b are advanced and deployed from extension posts 38a and 38b. One or both may be manipulated axially and/or rotatably to select and separate one or more chordae from the others. The arms are then rotated to a fully closed position to snugly surround the captured chordae. Device 30 is then advanced and tracked distally over the collected chordae to a location closer to the undersurface of the valve leaflets, thereby pulling the leaflet edges together to effect a coaptation between them. The suitability of the resulting apposition is then assessed, as described in greater detail below. If considered suitable, the clip 42 is advanced distally thereby allowing fastener legs 42a, 42b to spring close around the captured chordae 40. Arms 32a and 32b are then opened and, if applicable, retracted through posts 38a and 38b, respectively, leaving behind a secured fastener 42 around chordae 40, as shown in FIG. 3E. The procedure may be repeated as necessary to apply additional fasteners.

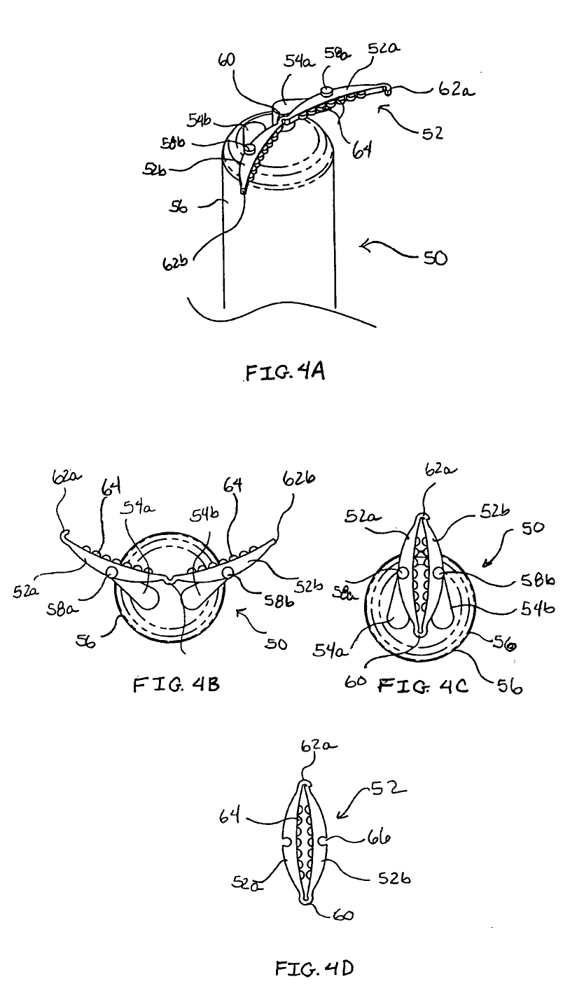

FIGS. 4A-4D illustrate another variation of a device of the present invention for repairing cardiac valves. A device 50 includes a main body 56, which may be a catheter, a cannula or the like, having a multi-functional member 52 positioned at a distal end for capturing a tissue structure, guiding the device to a target location and/or applying a fastening means at the target location. Member 52 is configured as a fastener or clip having arms or jaws 52a and 52b. Fastener 52 may be spring biased open (outwardly biased) or closed (inwardly biased) by means of a living hinge 60, but is not required to be so biased. Jaws 52a and 52b extend distally from hinge 60 to respective ends 62a and 62b which are configured to hold the jaws in a closed position when engaged. Any locking configuration may be used but may be as simple as a snap-fit closure, such as by means of a hook 62a and tab 62b. Atraumatic protrusions 64 extend laterally inward from opposing inner sides of jaws 52a and 52b to provide a snug yet atraumatic grip on a captured tissue structure, such as two or more opposing chordae (not shown). Lever arms 54a and 54b are pivotally mounted to the end of main body 56 for releasably holding jaw arms 52a and 52b, respectively, by means of mounting pins 58a and 58b, respectively, which are releasably received within respective holes 66 on the outer sides of the jaw arms. As with the arms 32a and 32b of device 30 of FIGS. 3A-3E, lever arms 54a and 45b are cooperatively operable by a user to be selectively opened and closed to affect the same motion on jaw arms 52a and 52b, respectively. Upon fully closing jaw arms 52a and 52b, enough force is exerted to lock hook 62 and tab 62b together. The force of the lock may be sufficient such that subsequent opening of lever arms 54a and 54b releases pins 58 from sockets 66. Alternatively, pins 58a, 58b may be released independently of the lever motion such as by a mechanism that retracts the pins out of sockets 66. In the illustrated embodiment, jaw movement is limited to within a plane perpendicular to the axis of main body 56; however, it is understood that the device may be configured wherein the jaws have a three-dimensional freedom of movement. Such a feature may be advantageous where it is desirable to attach the clip directly to the leaflets rather than to another tissue or prosthetic structure.

In use, device 50 is delivered to the target valve and the distal end of main body 56 is positioned proximate the valve leaflets, most typically in a subvalvular position. Again, particularly in endovascular applications, it may be beneficial to have clip arms 52a and 52b in a fully closed position upon delivery in order to minimize the device's profile. Upon delivery, jaw arms 52a and 52b are opened and one or both may be manipulated to select and separate one or more chordae from the others. The jaw arms are then caused to substantially close to surround the captured chordae. Device 50 is then advanced and tracked distally over the collected chordae to a location closer to the undersurface of the valve leaflets, thereby constricting the chordae and exerting tension on the leaflet edges to effect a coaptation between the leaflet edges. The suitability of the resulting apposition is assessed, as described in greater detail below. If considered suitable, the jaw arms are closed completely and locked together, and securely fastened clip 52 is released. The procedure may be repeated as necessary to apply additional fasteners.

While particular types of fastening means have been specifically illustrated and/or described, any suitable fastening means or the like and the means by which they are deployed at the target site may be used with the present invention with minor design changes to the fastener application/deployment mechanisms of the subject devices. Examples of fasteners and other means for use in attaching valve leaflets together and devices for delivery the fasteners, which may be employed with the devices and methods of the present invention, are disclosed in U.S. Pat. Nos. 6,165,183, 6,269,819, 6,312,447, 6,575,971, 6,629,534 and 6,641,593, and U.S. Patent Application Publication Nos. US 2002/0013571 and US 2003/0120341, the entireties of which are herein incorporated by reference. However, it is appreciated that such are only exemplary of the fastening means and fastening techniques that may be used in the context of the present invention, and that other fastening means and techniques not herein discussed or illustrated may be used.

The fastening means may be made of any suitable biocompatible material. Such biocompatible materials may be permanently implantable, i.e., not biodegradable. Representative permanently implantable materials include, but are not limited to, plastics such as RC-1008 plastic, commonly used by those skilled in the medical device arts, and metals or alloys thereof such as titanium, stainless steel, aluminum, Nitinol and the like.

The fastening devices may alternatively be made partially or wholly from bioresorbable or biodegradable materials such that they become absorbed or degrades at a rate that is sufficient to allow the angiogenic and arteriogenic processes to form tissue adhesion between the leaflets. Suitable biodegradable materials include, but are not limited to, polyurethane, poly (L-lactic acid), polycaprolactone, poly (lactide-co-glycolide), poly (hydroxybutyrate), poly (hydroxybutyrate-co-valerate), polydoxanone, polyorthoester, polyanhydride, poly (glycololic acid), poly (D,L-lactic acid), poly (glycololic acid-co-trimethylene carbonate), polyphosphoester, polyphosphoester urethane, poly (amino acids), cyanoacrylates, poly (trimethylene carbonate, poly (iminocarbonate), copoly (ether esthers) (e.g., PEO/PLA) polyalkylene oxalates, polyphosphazenes, as well as biomolecules such as fibrin, fibrinogen, cellulose, starch, collagen and hyaluronic acid.

The fastening means may also have the ability to diffuse drugs or other agents at a controllable rate at the valve leaflet coaptation or apposition site. One or more therapeutic agents may be added to the base material during fabrication of the fastener and/or a coating containing such therapeutic agents may be applied to the fastener after it has been fabricated. Suitable therapeutic agents for use with the subject fasteners include, but are not limited to, dexamethasone, tocopherol, dexamethasone phosphate, aspirin, heparin, coumadin, urokinase, streptokinase and TPA, or any other suitable thrombolytic substance to prevent thrombosis at or around the apposition point between the valve leaflets. Such therapeutic agents may be applied by spraying, dipping or other means. The subject fasteners may also be seeded with endothelial cells to promote angiogenesis between the fastener and the valve leaflet. Still further, the subject fasteners may include materials such as paralyne or other hydrophilic substrates that are biologically inert and reduce surface friction, where such materials may be applied by spraying, dipping or any other convenient means.

Furthermore, the fastening means may be configured to enable fluoroscopic visualization while delivering and operatively placing the fasteners on the valve leaflets. The fasteners may comprise one or more radio-opaque materials added to the fastener's base material during the fabrication process or a coating containing radio-opaque material may be applied to the fastener after it has been fabricated. Alternatively, the fasteners may be provided with one or more radiopaque markers. Any suitable material capable of imparting radio-opacity may be used, including, but not limited to, barium sulfate, bismuth trioxide, iodine, iodide, titanium oxide, zirconium oxide, metals such as gold, platinum, silver, tantalum, niobium, stainless steel, and combinations thereof.

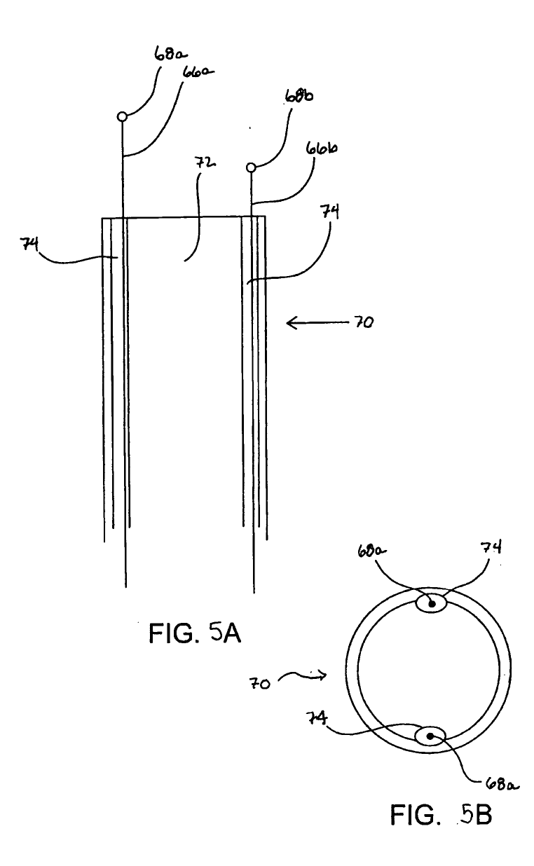

The various components described above including but not limited to a capturing or grasping device, a fastening tool, a fastener, flow probe, pressure monitor, etc. may be delivered to a target site by use of a delivery sheath 40 as illustrated in FIGS. 5A and 5B. Generally, sheath 70 has a proximal end, a distal end and at least one lumen 72 there between. The dimensions and material of such sheath 70 depend on the size of the fastener being deployed and the type of approach or access route a physician employs to access the target cardiac valve to be repaired.

For an endovascular approach, and cardiac valve applications in particular, a catheter is used as the delivery sheath 70. Catheters suitable for accommodating the fasteners of the present invention include those sized generally from about 6 to 30 French, but may be smaller or larger depending on the application and the intended delivery path to the target heart valve. Such catheters have lengths generally in the range from about 100 to 300 cm, but may be shorter or longer depending on the application and the intended delivery path to the target heart valve. Typically, the diameter of lumen 72 is sufficient to accommodate a fastener and associated fastener delivery device.

Materials suitable for use in the subject delivery catheters are chosen to provide the desired catheter flexibility and rigidity in order to manipulate the catheter through a patient's vasculature. The materials used to manufacture the catheter may also include radio-opaque materials, where such radio-opaque materials may include, but are not limited to, barium sulfate, bismuth trioxide, iodine, iodide, titanium oxide, zirconium oxide, gold, platinum, silver, tantalum, niobium, stainless steel, and combinations thereof.

In many embodiments of the devices of the present invention, including the tissue structure capturing device, the fastener delivery device, and delivery sheaths, the devices are steerable so that the clinician may temporarily impart a desired curve to the catheter from a remote location in order to be navigated within the patient's anatomy, e.g., through the patient's cardiovascular system. A variety of steering mechanisms known to those of skill in the art may be employed to impart the desired steerability. Generally, steerable catheters includes one or more pull wires which extend through the catheter shaft, and connect to the catheter adjacent the distal end of the catheter at an off-axis location. The pull wires connect to a control knob or knobs, slide actuator, or other suitable manipulating member that is mounted in a control handle. Representative catheters suitable for use with the subject invention include, but are not limited to, those used for electrophysiology, which are well known in the art.

For direct but less invasive or endoscopic approaches where the subject devices are delivered through a trocar port placed in the body, e.g., in the chest cavity, and delivered endoscopically to the target location, delivery sheath 70 is preferably a cannula. For cardiac valve applications, cannula 70 typically has a diameter in the range from about 4 to 12 mm, and more typically from about 6 to 8 mm, and lengths typically in the range from about 10 to 30 cm, and more typically from about 15 to 25 cm.

In either endovascular or endoscopic approaches, the catheter and cannula delivery devices of the present invention may further include additional lumens 74, as illustrated in FIGS. 5A and 5B, for delivering ancillary instrumentation for facilitating the implantation of the subject fasteners and clips. For example, these additional lumens 64 may be used to deliver pressure and/or flow monitoring probes 76a, 76b to the target valve to be repaired.

The monitoring element 78a of one probe 76a may be delivered to one side of the valve, e.g., within the right atrium to measure blood pressure just above the mitral valve, and a second monitoring element of 78b of the second probe 76b may be delivered to the other side of the valve, e.g., within the left ventricle to measure blood pressure just below the mitral valve. Alternately, a single probe having two spaced-apart monitoring elements may be used. With this alternate embodiment, the probe is delivered to a point where the distal monitoring element is positioned on the side of the valve opposite the delivery device and the proximal monitoring element is positioned on the side of the valve proximate the delivery device. With either embodiment, a pressure monitoring system (not shown) of the type known in the art external to the patient then measures the difference between the two pressures on opposing sides of the valve leaflets. Similarly, as mentioned above, a monitoring element may be positioned just above the mitral valve to measure back flow, if any, during systole. A variety of pressure monitoring probes and flow monitoring probes, which are known in the art, may be used with the subject invention. Additionally, other instrumentation, such as guide wires, endoscopes, and secondary grasping devices, may be delivered through additional lumens 74.

Also provided in the present invention are methods for repairing cardiac valves, e.g., mitral valves. In the subject methods, the target valve is access and the valve's leaflets are brought together at a first coaptation or apposition point along their edges, i.e., along the commissure line. Coaptation of mitral valve leaflets is accomplished by capturing one or more chordae attached to each leaflet and constricting the captured chordae so as to place tension on the leaflet edges. Alternatively, coaptation of the leaflets may be accomplished by capturing the leaflets themselves.

Once the leaflet motion is temporarily stabilized by the tension placed on them, the suitability of the selected apposition point is assessed by measuring one or more relevant characteristics related to the heart, such as blood flow and/or pressure, i.e., the modified or affected measurements, to verify the effectiveness of appositioning the leaflets together at this apposition point. Alternatively or additionally, TEE may be used to evaluate the effectiveness of the selected points of apposition. If the flow and/or pressure characteristics are not improved or are insufficient, the tension on the leaflets is removed by releasing the captured chordae, or the leaflets themselves, as the case may be. One or more different chordae, or the leaflets themselves, are then captured and constricted so as to appose the leaflets at another point, where such subsequent or second apposition point is similarly evaluated for suitability. Once an apposition point is determined suitable, the selected apposition point is fixed or permanently maintained by fastening together the captured chordae or leaflets. The subject steps may be repeated to successively apposition the leaflets at more than one selected apposition point along the commissure line (with or without permanency) until sufficient improvement in flow and/or pressure is achieved.

As mentioned above, the valve requiring repair may be access by means of an endovascular approach may be used which includes navigating a sheath such as a catheter through the vasculature of the patient and delivering a valve repair device there through, where the position of the catheter may be continuously verified by fluoroscopy and/or by transesophageal echocardiogram. Alternatively, a more direct approach may be used wherein the heart is accessed through a trocar port placed in the body, e.g., in the chest cavity and delivering a valve repair device through a sheath such as a cannula positioned through the port. Furthermore, while it is possible to perform the valve repair procedures described herein on a stopped heart, the procedures described herein are preferably performed on a beating heart, which will allow certain characteristics such as blood flow and/or pressure to be assessed during the procedure and eliminate the risks associated with cardiopulmonary bypass.

In those embodiments employing an endovascular or percutaneous approach to mitral valve repair using a sheath such as a catheter to access the heart, there exists two procedures which may be used: a retrograde approach and a transeptal approach. In the transeptal approach, the catheter is introduced into a patient's body percutaneously by means of a modified Seldinger technique via the right femoral vein. By means of transesophegeal echocardiogram, the catheter is then visualized, guided and advanced into the inferior vena cava and into the right atrium of the heart. The catheter then crosses the atrial septum through a small atrial septostomy (created by cardiological techniques known in the art) to enter the left atrium of the heart. For example, a guide wire may be placed across the atrial septostomy and the deliver catheter or a subject device as described above may then be threaded along the guide wire into the left atrium. The distal end or working end of the catheter can then be placed or brought to rest at a predetermined position in, at, or in proximity to the mitral valve.

In a retrograde endovascular approach, a delivery sheath or catheter or the like is introduced into a patient's body via a femoral artery. By means of transesophogeal echocardiogram visualization and guidance, the catheter or device is then advanced into the aorta, crossing the aortic valve into the left ventricle. The distal end or working end of the delivery catheter or the subject device can then be placed or brought to rest at a position in, at, or in proximity to the mitral valve, preferably at the underside of the mitral valve.

In those embodiments employing a direct access approach, the heart may be accessed by means of a traditional surgical approach, e.g., through a stemotomy, a thoracotomy, or a sub-xyphoid approach, or through one or more endoscopic ports positioned with in the chest cavity, e.g., between adjacent ribs. Once access to the heart is achieved, an entry site within a wall of the heart or a great vessel is created. More specifically, a penetrating means such as a trocar, obturator or guide wire or the like is used to penetrate the myocardium. If entry through the left ventricle or right ventricle is preferred for repair of the mitral valve and tricuspid valve, respectively, the apex of the heart is a suitable location to penetrate due to its resiliency to trauma. On the other hand, the entry site may be made in the wall of the left atrium or right atrium, respectively.

With any approach, the valve repair devices of the present invention may be delivered with or without the use of a delivery sheath catheter 70, as discussed above. Visualization and guidance of a delivery sheath and the other devices may be accomplished by transesophageal echocardiogram. Once the delivery sheath, such as the catheters or cannulas described above, is distally advanced and properly positioned in, at, or in proximity to the mitral valve, the blood flow and/or pressure gradient across the valve may be measured (although not required to be) such as by means of the pressure/flow monitoring devices described above, where such measurements may be used as baseline reference measurements. In other words, these measurements, i.e., one or both of pressure and flow measurements, may be made prior to capturing or grasping the chordae or valve leaflets so as to determine the base line or reference measurement of the blood flow and/or pressure gradient of the defective valve. Another set of measurement may be then be made after the chordae or leaflets have been grasped. The second measurement or sets of measurements, i.e., post-capture measurements, may then be compared to the first measurement or sets of measurements, i.e., pre-capturing, base line, or reference measurements, to determine the efficacy, i.e., the improvement on valve function, e.g., the reduction in regurgitation during systole, of appositioning the valve leaflets at the selected apposition point. Such comparison, i.e., the determination of the change in the pre- and post-grasping measurements is performed by a flow and/or pressure monitoring and control device, such as a microprocessor, operatively coupled to the proximal end of the one or more flow and/or pressure probes which extend proximally outside the patient's body. Alternatively or additionally, this determination may be made visually by means of TEE.

In those embodiments of the subject methods where baseline measurements are performed before the chordae or valve leaflets are captured or grasped, the delivery sheath is positioned adjacent either just above or below the leaflets of the valve to be repaired and flow and/or pressure monitoring probes are advanced out of the delivery catheter, i.e., out of one or more additional lumens of the delivery device to the target valve to be repaired. For example, a first pressure-monitoring element may be advanced to one side of the valve and a second pressure-monitoring element may be advanced to the other side of valve to measure the pressure on both sides of the valve during systole, i.e., the pressure differential or gradient across the valve may be measured during contraction of the heart. As described above, the pressure monitoring elements may be from a single probe, e.g., a single probe having spaced apart monitoring elements, or may be from different probes. In addition to or in place of the above described pressure measurement, a flow measuring element may also be advanced to the site of the target valve. More specifically, the flow probe is advanced out of the delivery device and positioned within the left atrium just above the valve leaflets and flow is measured during systole. As mentioned above, these measurements may be used as baseline or reference measurements against which to compare flow and/or pressure measurements taken after the leaflets have been brought together at one or more apposition points along their edges; however these pre-grasping measurements, i.e., the baseline measurements, may not and/or need not be performed in every instance.

Once baseline measurements are obtained, or in the case where baseline measurements are not first obtained, a repair device of the present invention, without or with the use of a delivery sheath 70, is delivered into the heart by any of the approaches discussed above, and is brought to a position in, at, or in proximity to the mitral valve, preferably the underside of the mitral valve leaflets. If the delivery approach includes transport through the left ventricle, the device may be tracked along the chordae tendinae as described above. The collected chordae are then synched or constricted, or the leaflets grasped, to affect an apposition point between the leaflets and provide at least some stability to the leaflet motion at that point. If the flow and/or pressure characteristics are being monitored, the necessary measurements may be made at this time, as described above, to determine the effectiveness of stabilizing the selected apposition point of the leaflets. With the leaflets being substantially stabilized, the collected chordae or valve leaflets may then be fastened together at one or more selected locations. The procedure may be repeated, if necessary, to place another fastener wherein a different chordae or set of chordae are employed to track the repair device to a selected location at the valve to be repaired.

The subject methods may further include the absorption or degradation of the subject fastener at a rate that is sufficient to allow the angiogenic and arteriogenic processes to form tissue adhesion between the leaflets. In other words, the fastener may break down after a set time period, during which time the apposition point of the leaflets is reinforced with vascularized tissue in-growth producing a sufficiently strong bond between the valve leaflets. Furthermore, one or more therapeutically relevant drugs or agents, discussed above, may be delivered or diffused to the defective valve and more specifically to the fastened apposition points, where such delivery or diffusion at a controlled rate by any convenient means discussed above.

Also provided by the subject invention are kits for use in practicing the subject methods. The kits of the subject invention at least include a tissue (e.g., chordae or leaflet) capturing device, as described above. The subject kits may also include one or more fasteners and associated delivery tools. The subject kits may further include one or more flow monitoring probes and/or one or more pressure monitoring probes. Furthermore, the subject kits may include additional instrumentation for performing the subject methods, where such additional instrumentation may include, but is not limited to, one or more guide wires, trocars, guide catheters, etc. Finally, the kits may further include instructions for using the subject fasteners and/or assemblies for repairing cardiac valves. The instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., CD-ROM, diskette, etc.

It is evident from the above description and discussion that the above described invention provides a device which, when operatively used, involves a simplified procedure by which to a repair cardiac valve, and, in particular, mitral and tricuspid valves. The above described invention provides a number of advantages, including the ability to temporarily apposition the valve leaflets and perform blood flow and/or pressure measurements while the leaflets are temporarily appositioned to verify whether the particular apposition point improves or optimizes flow and/or pressure before permanently appositioning the leaflets together. The subject invention also effectively corrects a defective valve in addition to other co-morbidities affecting proper function of the valve, obviating the need to perform ancillary procedures to correct leaflet size and shape, to reattach or shorten chordae, etc. Furthermore, the subject methods require minimal instrumentation and steps, is easier than conventional valve repair procedures to perform and reduces the time and cost of the procedure. As such, the subject invention represents a significant contribution to the art.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A method for repairing a cardiac valve having leaflets and subvalvular chordae, said method comprising:
(a) providing an apparatus configured for delivery to the cardiac valve, the apparatus comprising a means for capturing chordae;
(b) delivering the apparatus to a location proximate the valve;
(c) capturing chordae with the capturing means;
(d) appositioning the leaflets at a selected apposition point; and
(e) using the apparatus, applying a fastener to permanently appose the leaflets at a selected apposition site.

2. The method of claim 1, further comprising employing transesphogeal echo prior to applying the fastener in order to evaluate the effectiveness of the selected apposition point on the functioning of the valve.

3. The method of claim 1, further comprising measuring at least one of blood flow and pressure gradient across said valve prior to capturing the chordae wherein a baseline measurement is provided.

4. The method of claim 3, further comprising measuring at least one of blood flow and pressure gradient across said valve after capturing the chordae wherein an affected measurement is provided.

5. The method of claim 4, further comprising comparing the affected measurement to the baseline measurement to evaluate the effectiveness of the selected apposition point on the functioning of the valve.

6. The method of claim 5, wherein said steps (c) and (d) are repeated until the affected measurement indicates that the functioning of the valve leaflets is sufficiently improved.

7. The method of claim 1, wherein the capturing means comprises the fastener, wherein the fastener is releasable from the apparatus.

8. The method of claim 1, wherein the fastener is applied to the captured chordae.

9. The method of claim 1, wherein the fastener is applied to the leaflets.

8. The method of claim 1, wherein the method is performed by means of an endovascular approach.

9. The method of claim 1, wherein the method is performed by means of a surgical approach.

10. The method of claim 1, wherein the method is performed while the heart is beating.

11. The method of claim 1, further comprising, after said step (e), anchoring the fastener to a location on the cardiac anatomy

12. The method of claim 11, wherein the cardiac anatomy is the ventricle wall.

13. The method of claim 1, further comprising delivering at least one of a pressure monitoring probe and a flow monitoring probe proximate to the cardiac valve.

14. The method of claim 13, wherein the at least one of a pressure monitoring probe and a flow monitoring probe is delivered by means of the apparatus.

15. The method of claim 1, wherein at least one of the steps (b), (c), (d) and (e) is performed with the assistance of transesophageal echocardiogram.

16. The method of claim 1, wherein step (d) comprises tracking the device over the captured chordae.

17. A device for repairing a cardiac valve having leaflets and subvalvular chordae, the device comprising:
a main body configured for delivery to the cardiac valve;
a chordae capturing member associated with a distal end of the main body; and
a fastener application member associated with the distal end of the main body, wherein the fastener application member is used to apply a fastener to the valve anatomy to permanently appose the leaflets at a selected apposition site.

18. The device of claim 17, wherein the capturing member is selectively retractable within and extendable from the distal end of the main body.

19. The device of claim 17, wherein the fastener application member is selectively retractable within and extendable from the distal end of the main body.

20. The device of claim 17, wherein the capturing member comprises two rotatable arms.

21. The device of claim 20, wherein the two rotatable arms are simultaneously rotatable.

22. The device of claim 17, wherein the capturing member comprises a coiled loop.

23. The device of claim 17, wherein the capturing member has a low-profile configuration when retracted within the main body and has an expanded configuration when extended from the main body.

24. The device of claim 17, wherein the main body comprises a catheter.

25. The device of claim 17, wherein the main body comprises a cannula.

26. The device of claim 17, wherein the device is configured to deliver a fastener from outside a patient's body to proximate the valve leaflets.

27. The device of claim 26, wherein the device is configured to successively deliver a plurality of fastener.

28. The device of claim 27, wherein the device is configured to hold a cartridge of fasteners.

29. The device of claim 17, wherein the fastener application member comprises a lumen through which a fastener is delivered.

30. The device of claim 17, wherein the fastener application member is configured to restrain a fastener in an undeployed state.

31. A device for repairing a cardiac valve having leaflets and subvalvular chordae, the device comprising:
a main body configured for delivery to the cardiac valve; and
a mechanism at a distal end of the main body configured for capturing chordae and applying a fastener to the valve anatomy to permanently appose the leaflets at a selected apposition site.

32. The device of claim 31 wherein said mechanism comprises a releasable fastener.

33. The device of claim 32 wherein the mechanism further comprises means for selectively opening and closing the fastener.

34. The device of claim 33 wherein the fastener comprises two opposing jaws and wherein the means for opening and closing fastener comprises means for opening and closing the jaws.

35. The device of claim 34 wherein the fastener comprises means for locking the jaws together in a closed position.

36. The device of claim 35 wherein the mechanism is configured to controllably release the fastener from the device.

37. The device of claim 32 wherein the fastener is configured to capture the chordae.
